(19) United States
(12) Patent Application Publication
(10) Pub. No.: US 2012/0219590 A1
(43) Pub. Date: Aug. 30, 2012

(54) FORMULATIONS USEFUL FOR SPRAY DRYING VACCINES

(75) Inventors: Amish A. Patel, San Diego, CA (US); Paul W. Shabram, San Diego, CA (US)

(73) Assignee: Paxvax, Inc., San Diego, CA (US)

(21) Appl. No.: 13/404,950

(22) Filed: Feb. 24, 2012

Related U.S. Application Data
(60) Provisional application No. 61/446,309, filed on Feb. 24, 2011.

(51) Int. Cl. A61K 39/235; A61P 31/20; A61P 37/04; A61K 39/12; A61P 31/12

(52) U.S. Cl. 424/233.1; 424/204.1

(57) ABSTRACT

The present invention is directed to formulations for spray-drying viral particles, methods for spray drying such compositions, and pharmaceutical compositions and vaccines comprising the present spray-dried powders. The present formulations advantageously provide for spray-drying viral particles at low temperatures, thereby producing spray-dried viral powders having viral infectivities comparable to those of powders prepared by lyophilization of comparable formulations. The methods and compositions described herein advantageously provide substantially higher throughput and production rates for the production of viral powders. Further, spray-dried viral powders incorporating enteric polymers can be produced at low temperatures.

### CROSS REFERENCE TO RELATED APPLICATIONS

This application claims priority to U.S. Provisional Application Ser. No. 61/446,309 filed Feb. 24, 2011, which is hereby incorporated by reference in its entirety for all purposes.

### FIELD OF THE INVENTION

The present invention relates to preservation of biologically active materials. Specifically, the present disclosure relates to methods and compositions directed to spray drying viral particles and related products.

### BACKGROUND OF THE INVENTION

The preservation and storage of biologically active materials (e.g., viruses, proteins, cells, etc.) is important for medical and research purposes. Storage of dehydrated biologically active materials carries with it significant benefits. For example, dehydrated biological materials and cells typically have reduced weight and volume relative to solutions of such materials. Furthermore, such materials can, under certain conditions, have increased stability relative to their solution-phase counterparts. However, producing such dehydrated compositions comprising sensitive biological materials (e.g., thermally, chemically or mechanically unstable, etc.) can be problematic.

Conventional methods for producing stable preparations of sensitive biological materials in dehydrated form include freeze-drying and vacuum or air-drying. While freeze-drying methods are generally scalable for the production of commercially significant quantities of the desired materials, the materials dried by such methods typically can not be stored at ambient temperatures for long periods of time. In addition, the freezing step of freeze-drying can be damaging to many sensitive biological materials due to the mechanical stresses associated with freezing in aqueous solution, and subsequent sublimation of vapor. Alternatively, vacuum and air-drying methods generally are not scalable for the production of commercially significant quantities of the desired materials

Spray drying is another method for preserving sensitive biological samples. Spray drying avoids the stresses associated with freeze drying, and can provide similar performance as freeze drying in preserving the biological activity of dried samples. Laboratory scale spray-drying equipment typically allows researchers a great degree of flexibility due to the relatively low cost of equipment, minimal space requirements, however it provides low volume production. Normally conditions used in laboratory scale spray drying are not considered readily applicable to large scale production.

In addition, conventional spray-drying techniques and compositions often require relatively high (e.g., greater than about 70° C.) temperatures, which can degrade or otherwise attenuate the biological activity of a dried sample. Accordingly, there is a need for compositions suitable for spray-drying viral particles.

### SUMMARY OF THE INVENTION

In one aspect, the present invention is directed to formulations for spray-drying comprising viral particles, sucrose, and cyclodextrin. In another aspect, the present invention relates to a spray-dried powder comprising viral particles, sucrose and cyclodextrin. In still another aspect, the present invention relates to a formulation for spray drying comprising viral particles and a carbohydrate, wherein the total solids content of the formulation is less than about 30 wt %. In still another aspect, the present invention relates to a formulation for spray drying comprising viral particles, a carbohydrate and an enteric polymer. In various embodiments of the present invention, spray drying is conducted at an outlet temperature of less than about 40° C. In certain embodiments, the spray-dried powders of the present invention comprise viral particles wherein the viral activity of the viral particles is at least about 70% of the viral activity prior to spray drying. In still another aspect, the present invention relates to a pharmaceutical composition for oral administration comprising the present spray-dried powders. In still other embodiments, the present invention relates to vaccines comprising the present spray-dried powders.

The present invention advantageously provides formulations for spray-drying viral particles at low temperatures, thereby producing spray-dried viral powders having viral infectivities comparable to those of powders prepared by lyophilization (i.e., freeze-drying) of comparable formulations. The methods and compositions described herein advantageously provide substantially higher throughput and production rates for the production of viral powders. Further, spray-dried viral powders incorporating enteric polymers according to the present invention can be produced at low temperatures.

### BRIEF DESCRIPTION OF THE DRAWINGS

FIG. 1 is a flow chart showing comparative processes for the preparation of enteric coated viral particles using conventional lyophilization techniques versus spray drying.

FIG. 2 is an HPLC trace from analysis of an enteric coated spray dried virus powder re-suspended in buffer. The absorbance peak at 4.85 minutes corresponds to intact viral particles. The peaks surrounding the viral peak are seen with injection of the formulation buffer alone.

FIG. 3 is a schematic drawing of typical plant setup for spray drying.

### DETAILED DESCRIPTION

Throughout the present specification, the terms “about” and/or “approximately” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, +7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein. The terms “about” and “approximately” may be used interchangably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

Throughout the present specification, the words “a” or “an” are understood to mean “one or more” unless explicitly stated otherwise. Further, the words “a” or “an” and the phrase “one or more” may be used interchangeably.

In one aspect, the present invention relates to formulations for spray-drying comprising viral particles. The present formulations are generally aqueous formulations. Typically the formulations are prepared with purified water, e.g., deionized water or pharmaceutical grade water-for-injection (WFI). However, in some embodiments the present formulations may comprise water and one or more solvents which are miscible with water (e.g., ethanol, dimethyl sulfoxide, or any other pharmaceutically acceptable solvent or combination of solvents).

In certain embodiments, the present formulations are buffered. Buffering of the present formulations may provide desirable characteristics or properties, e.g., stabilization of the viral particles in the present formulations before and/or during and/or after spray-drying. Stabilization as used herein refers to the activity of the viral particles and the maintenance of the activity or immunogenic viability of viral particles. Suitable buffers may include any pharmaceutically acceptable buffering agents. For example, suitable buffering agents include phosphoric acid/phosphate salts, citric acid/citrate salts, HEPES and salts thereof, and combinations of pharmaceutically acceptable acids and/or salts, etc., In one embodiment, the present formulation is an aqueous formulation comprising mono- and dibasic sodium phosphate, and the formulation has a pH of about 7.5. The amount and/or concentration and/or identity of the buffering agent(s) employed may be selected to provide a desired level of pH stability of the present formulations. Typical concentrations of buffer in the present formulations may range from about 0.01 M to about 1M (e.g., about 0.1 M, about 0.2 M, about 0.3 M, about 0.4 M, about 0.5 M, about 0.6 M, about 0.7 M, about 0.8 M, about 0.9 M, or any other value or range of values therein). In one embodiment, the concentration of buffering agent is about 0.2 M. Suitable buffering agents and/or buffer concentrations and/or formulation pH levels may be selected to provide desired formulation properties. For example, the amount of buffer employed in the present formulations may be selected to provide an optimum pH level for stabilizing the viral particles.

Viral particles as described herein may be useful in vaccine applications. For example, viral particles in the present formulations may be any viral particles, e.g., adenoviral particles useful for vaccine or any other purposes. In some embodiments, the viral particles are adenoviral particles such as those described in co-pending U.S. patent application Ser. No. 12/847,767, filed on Jul. 30, 2010 (Atty. Docket No. PAXV-004/01US), the relevant portions of which are incorporated herein by reference. In some other embodiments, viral particles used for the present invention comprise an adenoviral vector that is replication competent. The present viral particles may be in any form, including live, live attenuated or killed, provided that the integrity of the antigenic determinant(s) is maintained.

The quantity of viral particles in the present formulations may be determined on the basis of the infectivity of spray dried powders produced from formulations comprising the viral particles. Accordingly, a target infectivity level or activity level may be obtained by increasing or decreasing the amount of viral particles in a formulation from which spray-dried powders comprising viral particles are produced. Viral particles spray dried in the present formulations typically have similar infectivities after spray drying. In some embodiments, viral particles after spray drying have at least 70%, at least 75%, at least 80%, at least 85%, at least 90%, at least 95%, or at least 98% (or any other value or range of values therein or thereabove) of infectivities or activities prior to spray drying.

The present formulations comprise one or more carbohydrates, which may stabilize the viral particles in the present formulations and in the spray-dried powders formed from the present formulations. Any physiologically and pharmaceutically acceptable carbohydrate may be used. For example, in certain embodiments, the present carbohydrate is a cyclodextrin (e.g., α-cyclodextirn, β-cyclodextrin, γ-cyclodextirn or mixtures thereof), which may be present with one or more other carbohydrates. In one embodiment, the carbohydrates are β-cyclodextrin and sucrose. In other embodiments, the carbohydrate(s) may include one or more of raffinose, mannitol, lactose, maltodextrin, sucrose, starch or combinations thereof. In some embodiments, the carbohydrate is maltodextrin. Suitable carbohydrates include, but are not limited to, monosaccharides, disaccharides, trisaccharides, oligosaccharides, polysaccharides, and their corresponding sugar alcohols, etc. Both natural and synthetic carbohydrates may be suitable for use in the present formulations.

Synthetic carbohydrates suitable for use in the present formulations include, but are not limited to, those which have the glycosidic bond replaced by a thiol or carbon bond. Both D and L forms of the carbohydrates may be used. The carbohydrate may be non-reducing or reducing. Exemplary reducing carbohydrates suitable for use in the present formulations include, but are not limited to, glucose, maltose, lactose, fructose, galactose, mannose, maltulose, iso-maltulose and lactulose. Exemplary non-reducing carbohydrates include, but are not limited to, trehalose, raffinose, stachyose, sucrose and dextran. Other useful carbohydrates may include non-reducing glycosides of polyhydroxy compounds selected from sugar alcohols and other straight chain polyalcohols. The sugar alcohol glycosides may be monoglycosides, e.g., compounds obtained by reduction of disaccharides such as lactose, maltose, lactulose and maltulose. Further, any of the above-described carbohydrate(s) may be hydrates.

The amount of carbohydrate in the present formulations may range from about 0.1 wt % to about 30 wt % (e.g., about 1 wt %, about 2 wt %, about 3 wt %, about 4 wt %, about 5 wt %, about 6 wt %, about 7 wt %, about 8 wt %, about 9 wt %, about 10 wt %, about 15 wt %, about 20 wt %, about 25 wt %, or any other value or range of values therein). In certain embodiments, the carbohydrates include β-cyclodextrin and sucrose, and the amount of β-cyclodextrin and sucrose in the formulation is about 5 wt %, about 6 wt %, about 7 wt %, about 8 wt %, about 9 wt %, about 10 wt %, about 11 wt %, about 12 wt %, about 13 wt %, about 14 wt %, about 15 wt %. In certain other embodiments, the carbohydrates include glucose and lactose, and the amount of glucose and lactose in the formulation is about 10 wt %. In certain other embodiments, the carbohydrates include raffinose and sucrose, and the amount of raffinose and sucrose in the formulation is about 11 wt %.

To improve the antigenicity of the viral particles, one or more adjuvants may be included in the mixture that is spray dried to create the present viral particle-containing powders. Examples of such adjuvants include, but are not limited to, salts, such as calcium phosphate, aluminum phosphate, calcium hydroxide, aluminum hydroxide and magnesium chloride; natural polymers such as algal glucans (e.g., beta glucans), chitosan or crystallized inulin, gelatin; synthetic polymers such as polylactides, polyglycolides, polylacitide coglycolides or methylacrylate polymers; micelle-forming cationic or non-ionic block copolymers or surfactants such as Pluronics, L121, 122 or 123, Tween 80, or NP-40; fatty acid, lipid or lipid and protein based vesicles such as liposomes, proteoliposomes, ISCOM and cochleate structures; stabilizers such as triethyl citrate, and surfactant stabilized emulsions composed of synthetic or natural oils and aqueous solutions.

Further, or to improve the properties of the present formulations or powders produced therefrom, one or more pharmaceutical excipients or other additives may be present in the present formulations. Such excipients or additives may include one or more stabilizing polyols, e.g., higher polysaccharides/polymers (for promoting controlled release), magnesium stearate, leucine and/or trileucine (as lubricants), and phospholipids and/or surfactants. Blowing agents, e.g., volatile salts such as ammonium carbonate, formic acid, etc. may also be included in the feedstock to produce reduced density particles in the present spray dried powders.

Spray aids may also be employed in the present formulation. Such spray aids may reduce the viscosity and/or improve the fluid mechanical characteristics of the present formulations during the spray drying process. Spray aids may include maltodextrin, lactose, gelatin, talc, triethylcitrate, and mixtures thereof. Such spray aids may be present in the formulations in amounts ranging from about 1 wt % to about 15 wt % (e.g., about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, or any other value or range of values therein). In certain embodiments, the spray aid is maltodextrin, and the amount of maltodextrin in the formulation is about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %. In other embodiments, the spray aid is lactose, and the amount of lactose in the formulation is about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %. In still other embodiments, the spray aid is gelatin, and the amount of gelatin in the formulation is about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %.

Surfactants may also be employed in the present formulations. For example, surfactants employed may include polysorbates, poloxamers and mixtures thereof. The surfactant(s) may comprise from about 0.01 wt % to about 2 wt % of the present formulations (e.g., about 0.02 wt %, about 0.04 wt %, about 0.06 wt %, about 0.08 wt %, about 0.1 wt %, about 0.2 wt %, about 0.3 wt %, about 0.4 wt %, about 0.5 wt %, about 1.0 wt %, about 1.5 wt %, or any other value or range of values therein).

In some embodiments, the present formulations may comprise one or more enteric polymers. Spray drying of formulations comprising an enteric polymer allows simultaneous drying and coating of viral particles. Accordingly, the present formulations may comprise from about 0.1 wt % to about 5 wt % of an enteric polymer (e.g., about 0.2 wt %, about 0.4 wt %, about 0.6 wt %, about 0.8 wt %, about 1 wt %, about 2 wt %, about 3 wt %, about 4 wt %, or any other value or range of values therein). Representative examples of enteric polymers which may be useful in the present formulations include esters of cellulose and its derivatives (e.g., cellulose acetate phthalate, hydroxypropyl methylcellulose phthalate, hydroxypropyl methylcellulose acetate succinate), polyvinyl acetate phthalate, pH-sensitive methacrylic acid-methacrylate copolymers and shellac. In one embodiment, the enteric polymer is Eudragit® L 30 D-55. In certain embodiments, the enteric polymer is Eudragit® L 30 D-55, and is present in the formulation at about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %. In some embodiments, the enteric polymer is hydroxypropyl methylcellulose phthalate, and is present in the formulation at about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %. In other embodiments, the enteric polymer is cellulose acetate phthalate, and is present in the formulation at about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %. Enteric polymers are widely used in the pharmaceutical industry for encapsulation of an active drug, for taste masking, and/or for immediate and/or modified release formulations, either alone or in combination with one or more other polymers (e.g., water insoluble polymers, water soluble polymers, etc.). Modified-release formulations may include, e.g., delayed-release, extended-release, site-specific targeting and receptor targeting. Such modified release formulations are known and will be appreciated by those skilled in the art.

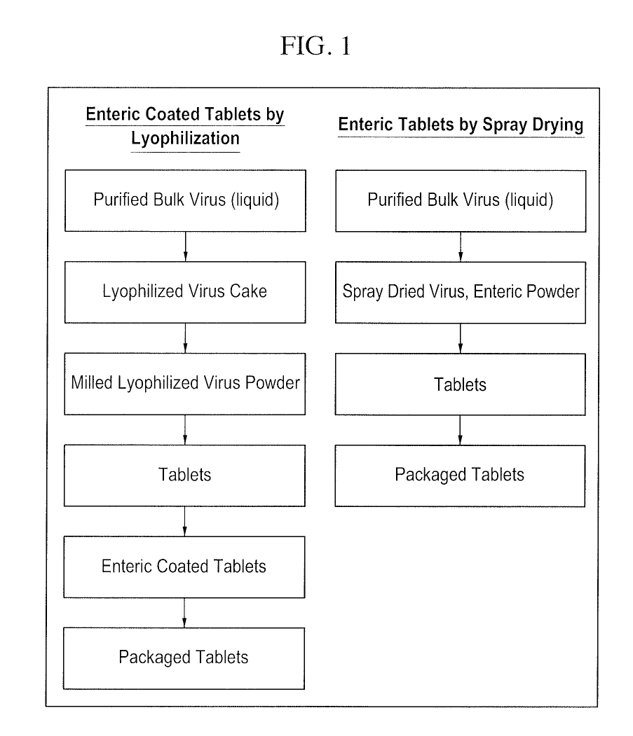

Eudragit® and/or other comparable enteric polymers as described herein are available as aqueous or organic solutions or as powders. The use of enteric or other suitable polymers may facilitate the formation of spray dried powders wherein viral particles and any additives and/or excipients are encapsulated or coated with an enteric polymer to yield a free-flowing and non-dusty powder suitable for direct tablet compression. Accordingly, enteric coating of virus particles by spray-drying of formulations comprising an enteric polymer may have several advantages in an exemplary dosage manufacturing process. For example, as shown in FIG. 1, a production process for an enteric coated product may be greatly simplified by spray-drying formulations comprising viral particles and an enteric polymer, rather than freeze-drying viral particles, forming tablets therefrom, and then applying an enteric coating thereto. As shown in FIG. 1. spray drying formulation comprising enteric polymer may also reduce the number of steps and unit operations in the production of enteric coated dosage forms.

One exemplary formulation comprises carbohydrates sucrose and fructose, spray aid gelatin, glycerin, surfactant poloxamer, phosphate buffer and magnesium chloride. Another exemplary formulation comprises carbohydrates sucrose and β-cyclodextrin, spray aid maltodextrin, glycerin, a surfactant mixture of polysorbate and poloxamer, phosphate buffer and magnesium chloride concentration of about 10 mM. Still another exemplary formulation comprises galactose and β-cyclodextrin, spray aid talc, glycerin, polysorbate, phosphate buffer and magnesium chloride.

One exemplary formulation comprises about 10 wt % sucrose and β-cyclodextrin, about 7 wt % maltodextrin, about 0.4 wt % glycerin, from about 0.2 to about 0.5 wt % polysorbate, a phosphate buffer concentration of about 200 mM and a magnesium chloride concentration of about 10 mM. Another exemplary formulation comprises about 12 wt % sucrose and β-cyclodextrin, about 5 wt % maltodextrin, about 4 wt % glycerin, from about 0.2 to about 0.5 wt % polysorbate, a phosphate buffer concentration of about 200 mM and a magnesium chloride concentration of about 10 mM. Still another exemplary formulation comprises about 14 wt % sucrose and β-cyclodextrin, about 3 wt % maltodextrin, about 0.4 wt % glycerin, from about 0.2 to about 0.5 wt % polysorbate, a phosphate buffer concentration of about 200 mM and a magnesium chloride concentration of about 10 mM.

One exemplary formulation comprises about 2 wt % sucrose and β-cyclodextrin, about 14 wt % maltodextrin, about 0.4 wt % glycerin, from about 0.2 to about 0.5 wt % polysorbate, about 0.4 wt % Eudragit® L 30 D-55, a phosphate buffer concentration of about 200 mM and a magnesium chloride concentration of about 10 mM. Another exemplary formulation comprises about 4 wt % sucrose and β-cyclodextrin, about 12 wt % maltodextrin, about 0.4 wt % glycerin, from about 0.2 to about 0.5 wt % polysorbate, about 0.5 wt % Eudragit® L 30 D-55, a phosphate buffer concentration of about 200 mM and a magnesium chloride concentration of about 10 mM. Another exemplary formulation comprises about 6 wt % sucrose and β-cyclodextrin, about 10 wt % maltodextrin, about 0.4 wt % glycerin, from about 0.2 to about 0.5 wt % polysorbate, about 0.6 wt % Eudragit® L 30 D-55, a phosphate buffer concentration of about 200 mM and a magnesium chloride concentration of about 10 mM.

The total solids content of the present formulations may range from about 5 wt % to about 30 wt % (e.g., about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, or any other value or range of values therein). In some embodiments, the total solids content is less than about 30 wt %. In other embodiments, the total solids content is less than about 10 wt %.

The present formulations are generally prepared by sequentially adding and dissolving each component in solution (e.g., to WFI or DI water), mixing the resultant solution until a given component is dissolved, then adding the next component. However, in certain embodiments, it may be advantageous to pre-mix certain components separately in two or more solutions, then combine the two or more solutions to obtain a final formulation for subsequent spray-drying. Alternatively, it may be advantageous to add the various components of the mixture in a certain order such that the resultant solution is substantially homogenous.

Particles produced from spray drying the present formulations will generally have a composition which reflects the proportions of non-volatile components in the spray drying solution from which they were formed. Particles produced from the present formulations will have a composition which corresponds to that of the formulation form which they are spray dried, less any volatile components removed during the spray drying process (e.g., water). Thus, in one embodiment, the present spray-dried powders comprise viral particles, sucrose and cyclodextrin. In another embodiment, the present spray-dried powders comprise viral particles, a carbohydrate, and an enteric polymer. Particles produced by spray drying the present formulations will also be substantially homogeneous, both within a particle and from particle to particle. Particle morphology is generally spherical. In some embodiments, the present particles may be substantially solid. In other embodiments, the particles may be hollow spheres. Morphology of the present particles may be controlled by adjusting various spray drying parameters and/or formulation parameters, as known to those skilled in the art.

Particles produced from the present formulation may have a size ranging from about 1 μm to about 500 um, (e.g., from about 1 μm to about 400 μm, from about 1 μm to about 300 μm, from about 1 μm to about 200 μm, from about 1 μm to about 100 μm, from about 2 μm to about 50 μm, from about 2 μm to about 20 μm, from about 2 μm to about 10 μm, or any other value or range of values therein). When powders produced by according to the present invention are intended for pulmonary delivery, particles having a mean diameter ranging from about 0.1 μm to about 10 μm may be employed for deep lung deposition. Alternatively, if the powder is intended for nasal delivery, particle diameter may be larger, e.g., from about 10 μm to about 75 μm. Particle size in the present powders may be measured, for example, using an Horiba LA-950 laser diffraction dry powder feeder apparatus or an equivalent means of measuring.

Furthermore, particle sizes may reported as a statistical distribution. For example, where particle diameter is reported as about 15 μm, that measurement may reflect some fraction of the total distribution having a particle size of about 15 μm. Thus, the a distribution or “d” value may report some percentage of the whole having the reported size. For example, a d80 value of 15 μm particle diameter reports that approximately 80% of the particles measured have a diameter of about 15 μm. Similarly, d70, d75, d85, d90, d95 (or any other value or range of values therein or thereabove) values, may be used. Such determination of particle size and statistical distributions associated therewith are known and will be appreciated by those skilled in the art.

The present formulations may advantageously be spray-dried at lower temperatures relative to conventional compositions for spray-drying viral particles. Thus, in some embodiments, the present formulations are spray dried with an inlet temperature of less than about 70° C., less than about 60° C., less than about 50° C., less than about 40° C., less than about 35° C., less than about 30° C., or any other value or range of values therein or therebelow. Accordingly, in certain embodiments, the present formulations are spray dried with an outlet temperature of less than about 50° C., less than about 40° C., less than about 30° C., less than about 25° C., less than about 20° C., or any other value or range of values therein or therebelow. Such reduced inlet and outlet temperatures advantageously enable spray-drying of viral particles without significant loss of viral infectivity upon drying.

The present formulations are also suitable for large-scale production of spray dried viral powders with, e.g., commercial spray-drying equipment. Thus, in certain embodiments, the present formulations may be spray dried at production rates of at least about 50 g/hr, about 60 g/hr, about 70 g/hr, about 80 g/hr, about 90 g/hr, about 100 g/hr, about 120 g/hr, about 140 g/hr, about 160 g/hr, about 180 g/hr, about 200 g/hr, about 220 g/hr, about 240 g/hr, about 260 g/hr, about 280 g/hr, about 300 g/hr, about 350 g/hr, about 400 g/hr, about 450 g/hr, about 500 g/hr, or any other value or range of values therein or thereabove. Accordingly, in certain embodiments, the present methods provide large-scale drying of the present formulations at reduced temperatures (e.g., less than about 40° C.) as previously described herein.

Particles produced from the present formulations may be spray dried such that they have a specific moisture content after spray drying. For example, the present spray dried powders may have a moisture content of less than about 10 wt % (e.g., less than about 9 wt %, less than about 8 wt %, less than about 7 wt %, less than about 6 wt %, less than about 5 wt %, less than about 4 wt %, less than about 3 wt %, less than about 2 wt %, less than about 1 wt %, or any other value or range of values therein or therebelow).

It will be appreciated by the skilled person that the present spray-dried particles are to be formulated to contain physiologically effective amounts of a virus. That is, when the present spray dried powders are delivered in a unit dosage form, there should be a sufficient amount of viral particles to achieve a desired therapeutic response.

In some embodiments of the present invention, spray dried powders comprising viral particles produced from the present formulations may be used directly. In other embodiments, the present powders may be used as a component in the preparation of vaccines. Accordingly, the present invention provides vaccines comprising one or more viral vectors in the present spray-dried powders. In certain embodiments, the viral vector is an adenoviral vector. In some other embodiments, the viral vector is a virus. In yet other embodiments, the viral vector includes the viral genome alone and does not include a viral capsid.

In certain embodiments, a vaccine of the invention, upon administration to a subject, is capable of stimulating an immune response (e.g., an innate immune response, humoral immune response, cellular immune response, or all three) in the subject. In certain embodiments, the immune response includes a measurable response (e.g., a measurable innate, humoral or cellular immune response, or combination thereof) to an epitope encoded by a heterologous sequence inserted or integrated into an adenoviral vector of the vaccine. In certain embodiments, a vaccine of the invention is capable of providing protection against an infectious pathogen or against cancer. For example, in certain embodiments, the vaccine is capable of stimulating an immune response against one or more antigens (e.g., encoded by a heterologous sequence) such that, upon later encountering such an antigen, the subject receiving the vaccine has an immune response that is stronger than it would have been if the vaccine had not been administered previously.

In some embodiments, a vaccine of the invention is capable of providing protection against an infectious pathogen or cancer in a subject with pre-existing immunity to adenovirus. In other embodiments, a vaccine of the invention is capable of ameliorating a pathogen infection or cancer and/or reducing at least one symptom of a pathogen infection or cancer. For instance, in one embodiment, the vaccine of the invention induces a therapeutic immune response against one or more antigens encoded by a heterologous sequence such that symptoms and/or complications of a pathogen infection or cancer will be alleviated, reduced, or improved in a subject suffering from such an infection or cancer.

The present vaccines can be prepared and formulated as a pharmaceutical composition for administration to a mammal in accordance with techniques well known in the art. Formulations for oral administration can consist of capsules or tablets containing a predetermined amount of the present spray-dried powders; liquid solutions comprising the present spray-dried powder dissolved in an ingestible diluent such as water, saline, orange juice, and the like; suspensions in an appropriate liquid; and suitable emulsions.

The present vaccines can, for example, be formulated as enteric coated capsules or tablets for oral administration in order to bypass the upper respiratory tract and allow viral replication in the gut. See, e.g., Tacket et al., Vaccine 10:673-676, 1992; Horwitz, in Fields et al., eds., Fields Virology, third edition, vol. 2, pp. 2149-2171, 1996; Takafuji et al., J. Infec. Dis. 140:48-53, 1979; and Top et al., J. Infec. Dis. 124:155-160, 1971. Alternatively, in the case of powders comprising enteric coated viral particles as previously described herein, such enteric powders may be directly compressed to provide oral, enteric coated formulations. Alternatively, the vaccine can be formulated as a pharmaceutical composition in conventional solutions, such as sterile saline, and may incorporate one or more pharmaceutically acceptable carriers or excipients. The pharmaceutical composition can further comprise other active agents.

In certain embodiments, formulations of the invention comprise a buffered solution comprising the present spray-dried powders in a pharmaceutically acceptable carrier. A variety of carriers can be used, such as buffered saline, water and the like. Such solutions are generally sterile and free of undesirable matter. These compositions can be sterilized by conventional, well known sterilization techniques, or can be sterile filtered. The compositions may contain pharmaceutically acceptable auxiliary substances as required to approximate physiological conditions such as pH adjusting and buffering agents, tonicity adjusting agents and the like, for example, sodium acetate, sodium chloride, potassium chloride, calcium chloride, sodium lactate and the like.

Pharmaceutically acceptable carriers can contain a physiologically acceptable compound that acts, e.g., to stabilize the composition or to increase or decrease the absorption of the virus and/or pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of any co-administered agents, or excipient, or other stabilizers and/or buffers. Detergents can also be used to stabilize the composition or to increase or decrease absorption. One skilled in the art will appreciate that the choice of a pharmaceutically acceptable carrier, including a physiologically acceptable compound depends, e.g., on the route of administration of the present powders and on the particular physio-chemical characteristics of any co-administered agent.

The adenoviral vectors can also be administered in a lipid formulation, more particularly either complexed with liposomes or to lipid/nucleic acid complexes or encapsulated in liposomes. The vectors of the current invention, alone or in combination with other suitable components, can also be made into aerosol formulations to be administered via inhalation. The vaccines can also be formulated for administration via the nasal passages. Formulations suitable for nasal administration, wherein the carrier is a solid, include a coarse powder having a particle size, for example, in the range of about 10 to about 500 microns which is administered in the manner in which snuff is taken, e.g., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. In some embodiments, vaccines of the invention can be formulated as suppositories, for example, for rectal or vaginal administration.

Vaccines can have a unit dosage comprising between about 10³ to about 10¹³ (e.g., about 10³ to about 10⁴, about 10⁴ to about 10⁵, about 10⁵ to about 10⁶, about 10⁶ to about 10⁷, about 10⁷ to about 10⁸, about 10⁸ to about 10⁹, about 10⁹ to about 10¹⁰, about 10¹⁰ to about 10¹¹, about 10¹¹ to about 10¹², about 10¹² to about 10¹³) recombinant adenoviruses in a single dose. The dosages can vary based on the route of administration. For instance, vaccines formulated for sublingual or intranasal administration may contain a lower dosage of adenovirus per single dose than vaccines formulated for oral administration. One of skill in the art can determine the appropriate dosage for a particular patient depending on the type of infection or cancer, and the route of administration to be used without undue experimentation.

The vaccines of the invention can be administered alone, or can be co-administered or sequentially administered with other immunological, antigenic, vaccine, or therapeutic compositions. Such compositions can include other agents to potentiate or broaden the immune response, e.g., IL-2 or other cytokines which can be administered at specified intervals of time, or continuously administered (see, e.g., Smith et al., N Engl J Med 1997 Apr. 24; 336(17):1260-1; and Smith, Cancer J Sci Am. 1997 December; 3 Suppl 1:S137-40). The vaccines or vectors can also be administered in conjunction with other vaccines or vectors. For example, a vaccine of the invention can be administered either before or after administration of an adenovirus of a different serotype. A vaccine preparation may also be used, for example, for priming in a vaccine regimen using an additional vaccine agent.

The present vaccines can be delivered systemically, regionally, or locally. Regional administration refers to administration into a specific anatomical space, such as intraperitoneal, intrathecal, subdural, or to a specific organ, and the like. Local administration refers to administration of a composition into a limited, or circumscribed, anatomic space such as an intratumor injection into a tumor mass, subcutaneous injections, intramuscular injections, and the like. Those skilled in the art will appreciate that local administration or regional administration can also result in entry of the viral preparation into the circulatory system. Typical delivery routes include parenteral administration, e.g., intradermal, intramuscular or subcutaneous routes. Other routes include oral administration, including administration to the oral mucosa (e.g., tonsils), intranasal, sublingual, intravesical (e.g., within the bladder), rectal, and intravaginal routes. For delivery of adenovirus, administration can often be performed via inhalation. Aerosol formulations can, for example, be placed into pressurized, pharmaceutically acceptable propellants, such as dichlorodifluoro-methane, nitrogen and the like. They can also be formulated as pharmaceuticals for non-pressurized preparations such as in a nebulizer or an atomizer. Typically, such administration is in an aqueous pharmacologically acceptable buffer as described above. Delivery to the lung can also be accomplished, for example, using a bronchoscope.

The vaccines of the invention can be administered in a variety of unit dosage forms, depending upon the intended use, e.g., prophylactic vaccine or therapeutic regimen, and the route of administration. With regard to therapeutic use, the particular condition or disease and the general medical condition of each patient will influence the dosing regimen. The concentration of adenovirus in the pharmaceutically acceptable excipient can be, e.g., from about 10³ to about 10¹³ virus particles per dose, between about 10⁴ to about 10¹¹ virus particles per dose, between about 10⁶ to about 10¹⁰ virus particles per dose, between about 10⁷ to about 10⁹ virus particles per dose, or between about 10⁹ to about 10¹¹ virus particles per dose. In other embodiments, the concentration of adenovirus in the pharmaceutically acceptable excipient can be, e.g., from about 10³ to about 10⁹, about 10⁴ to about 10⁸, or about 10⁵ to about 10⁷ infectious units per dose.

The replication-competent adenoviral vectors of the invention are typically administered at much lower doses than would be needed to achieve equivalent expression levels of the encoded transgene by a replication-defective adenovirus recombinant administered in vivo. Replication competent adenovirus vectors can be administered at a range of dosages (see, e.g., U.S. Pat. No. 4,920,209; Smith et al., J. Infec. Dis. 122:239-248, 1970; Top et al., J. Infect. Dis. 124:155-160, 1971; Takafuji et al., J. Infec. Dis. 140:48-53, 1979; Tacket et al., Vaccine 10:673-676, 1992). For example, 10⁴ to 10⁹50% tissue culture infective doses (or plaque forming units) can be administered. Typically an oral dosage for a replication-competent adenovirus is about 10⁴⁶ 50% tissue culture infective doses or 10⁷ pasticles. In some embodiments, an oral dosage for a replication-competent adenovirus is about 10¹¹ particles. Typical intranasal administration of adenovirus recombinants is often in dosages of about 10³ to about 10⁵ plaque forming units. The exact concentration of virus, the amount of formulation, and the frequency of administration can also be adjusted depending on the levels of in vivo, e.g., in situ transgene expression and vector retention after an initial administration.

The amount and concentration of virus and the formulation of a given dose, or a “therapeutically effective” dose can be determined by the clinician. A therapeutically effective dose of a vaccine is an amount of adenovirus that will stimulate an immune response to the protein(s) encoded by the heterologous nucleic acid included in the viral vector. The dosage schedule, i.e., the dosing regimen, will depend upon a variety of factors, e.g., the general state of the patient's health, physical status, age and the like. The state of the art allows the clinician to determine the dosage regimen for each individual patient. Adenoviruses have been safely used for many years for human vaccines. See, e.g., Franklin et al., supra; Jag-Ahmade et al., J. Virol., 57:267, 1986; Ballay et cd., EMBO J. 4:3861, 1985; PCT publication WO 94/17832. These illustrative examples can also be used as guidance to determine the dosage regimen when practicing the methods of the invention.

Single or multiple administrations of adenoviral formulations can be administered as prophylactic or therapeutic vaccines. In one embodiment, multiple doses (e.g., two or more, three or more, four or more, or five or more doses) are administered to a subject to induce or boost a protective or therapeutic immune response. The two or more doses can be separated by periodic intervals, for instance, one week, two week, three week, one month, two month, three month, or six month intervals.

The following non-limiting examples will illustrate various aspects of the present invention. The examples should, of course, be understood to be merely illustrative of only certain embodiments of the invention and not to constitute limitations upon the scope of the invention which is defined by the claims that are appended at the end of this description.

### EXAMPLES

### Viral Infectivity

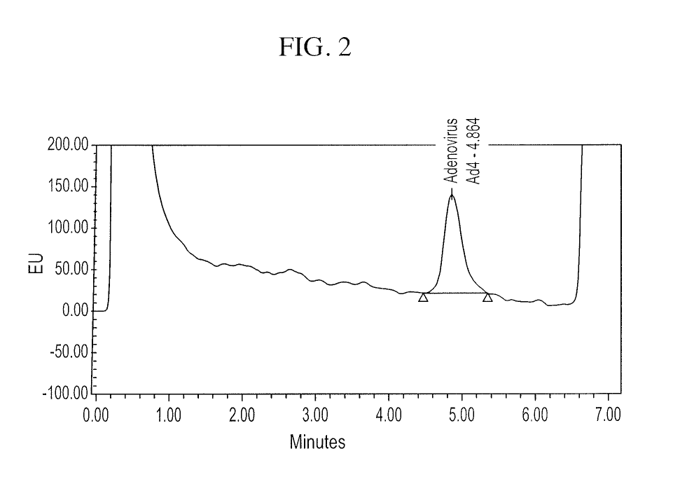

To evaluate the quality of and infectivity of viral powders produced by spray drying relative to those produced by lyophilization, two different types of assays for determining the viral titer are employed: 1) Viral Particle Titer by Anion Exchange HPLC method (AE-HPLC, see FIGS. 2); and 2) Infectious Titer by NAS-TCID₅₀ assay based on the viral infection of cells. The ratio of the results of these assays (vp/NVIU) can be used to qualitatively evaluate the spray-dried material for infectivity. Table 1 below summarizes the infectivity ratio of different formulations and drying methods. As can be seen from the data in table 1 below, spray dried powders of the present invention have vp/NVIU values on the same order of magnitude as those obtained by lyophilization.

TABLE 1
Viral particle-to-Infectivity ratio for different formulations.

| | Viral Particles | Infectious Titer by | |
|---|---|---|---|
| | by AE-HPLC | NAS TCID₅₀ | vp/NVIU |
| Formulation | (vp/mg) | (NVIU/mg) | ratio |
| Lyophilized Powder 1 | 3.90E+07 | 1.18E+05 | 331 |
| Lyophilized Powder 2 | 6.23E+07 | 1.76E+05 | 354 |
| Spray Dried Non | 6.96E+06 | 3.06E+04 | 228 |
| Enteric coated | | | |
| Powder Formulation 9 | | | |
| Spray Dried Enteric | 1.47E+07 | 5.58E+04 | 263 |
| coated Powder | | | |
| Formulation 10 | | | |

### Placebo Formulation Testing

Spray drying of placebo formulations was performed to decrease the outlet temperatures below 40° C., and the optimized conditions and formulations and spray drying process parameters were employed in drying adenovirus-containing compositions.

Thirty-one runs were performed in nine days of testing on the SD-Micro spray dryer. In general, the testing went well, although some of the formulations and excipients did not produce favorable spray drying results. In general, an outlet temperature as low as 21 degrees C. was shown to be able to produce good powders with formulations comprising maltodextrin, sucrose, and β-cyclodextrin. Yields in the 50% range were demonstrated for suspensions containing Eudragit L30-D55 using the 21 degree outlet temperature. Suspensions containing mannitol instead of maltodextrin did not work well (sticky), even with the addition of talc. The one formulation which contained gelatin produced the highest yield of the testing (56%). Suspensions containing milk were not successful.

### Equipment and Process Setup

Niro SD-Micro Spray Dryer (S/N 090-0025-1-01)
Insulated Stainless Steel Drying Chamber, Baghouse, cyclone, and ductwork
Niro two-fluid nozzle w/0.5 mm liquid insert
2.4 mm ID, 3.2 mm OD Marprene tubing (no “Y”)
Watson Marlow 505 L Peristaltic Pump
Filter cleaning=manual
30 LPM Nitrogen purge on cyclone collection jar

Compressed nitrogen was used as the drying gas with flammable solutions or solutions requiring a “dry” drying gas and was heated electrically. The drying gas entered the top of the drying chamber through a ceiling gas distributor. The feed was transferred to the two-fluid nozzle (TFN) at the top of the chamber through tubing by means of a pneumatic peristaltic pump. From the drying chamber the particle-laden air flowed through a cyclone for primary particle separation. The primary particles are collected in glass media bottles. After the cyclone, the particle-laden gas (very fine particles not separated by the cyclone) flowed to a bag filter for secondary particle separation. After the bag filter the humid gas was released to atmosphere.

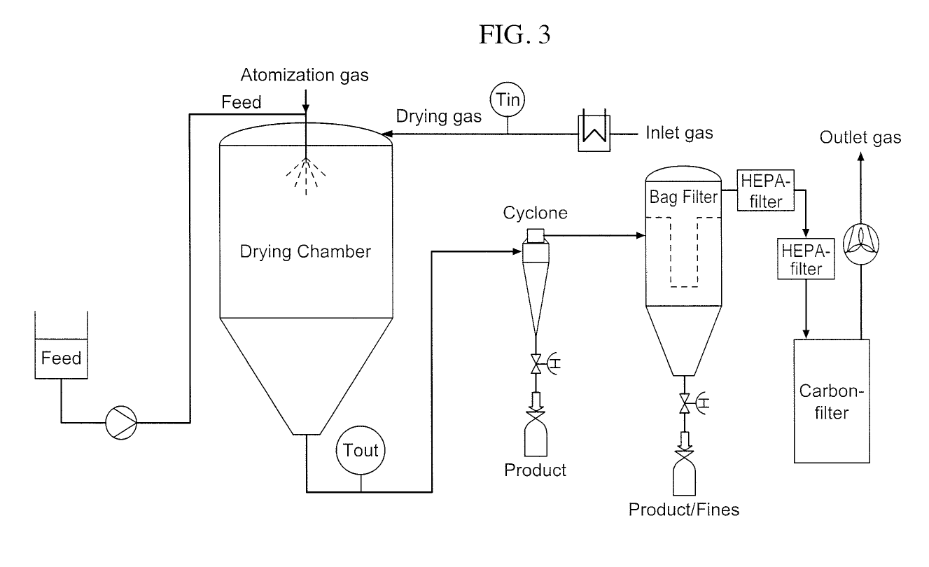

FIG. 3 shows a schematic drawing of typical of the plant setup that was used in these trials. The SD-Micro was selected based on the quantities of material available, and the ability to use nitrogen as the process gas. The most common reason to use nitrogen as the drying gas is for processing organic solvents or for product stability concerns. Using Nitrogen in a single-pass configuration (see FIG. 3) has the additional benefits of yielding low residual water levels in the powders that are produced, as the inlet compressed Nitrogen has a very low dew point. The Two-Fluid Nozzle was selected due to the laboratory scale of spray drying, and the ease of use and adjustability.

A Nitrogen purge on the cyclone collection jar was installed for this test to emulate the conditions for the future test work with live virus. The Nitrogen was regulated at 2.0 psig and 30 LPM, and was approximately 18 degrees C. with a dewpoint of −12 degrees C. For this testing, the ductwork and cyclone were insulated with piping insulation (McMaster Carr).

Testing began with the formulations containing maltodextrin, and efforts were made to reduce the outlet temperature. Powders were successfully produced at outlet temperatures as low as 21 degrees C. Preliminary data which indicates that the virus survival rate is dependent on the outlet temperature, with differences in the survival rate detectable even between 35 and 30 degrees C. Therefore it was desired to demonstrate that the spray drying was feasible as low as 20 degrees C. The reduced throughput in the spray dryer is acceptable if the virus survival rate is improved as a result.

Once the low temperature was successfully demonstrated, the atomization was varied to try and improve the process yield. At 3.0 kg/hr, the yield was over 40%, which was acceptable, and therefore it was used for the majority of the remaining test work. The total solids content was lowered to around 10%, and the process worked more smoothly. Tween concentration was doubled from Run 4 to Run 5, and in Run 6 the maltodextrin was replaced with sucrose. In Runs #12-17, mannitol was used instead of maltodextrin. The equipment was very sticky and it was determined that mannitol did not work at the low-temperature conditions which were being used.

Runs #22-28 contained Eudragit, and produced very nice, free-flowing powders. It is highly desirable to include Eudragit, as enteric coating the powders would eliminate the need to enteric coat either the capsules currently being used, or ultimately a tablet.

Feed Formulations used for the testing are listed in tables 2-5, below. All feed preparation work was performed using magnetic stir plates and typical laboratory supplies. The process parameters employed are listed in tables 6-7, below. All samples were collected in 100 mL Kimax media bottles.

TABLE 2
Placebo Feed Formulations 1-10

| Formulation No. | 1 | 2 | 3 | 4 | 5 | 6 | 7 | 8 | 9 | 10 |
|---|---|---|---|---|---|---|---|---|---|---|
| 2% milk 9% solid ml | | | | | | | | | | |
| wfi water ml | 110 | 110 | 670 | 238 | 110 | 110 | 110 | 110 | 110 | 110 |
| 1M mgcl2 ml | 2.5 | 2.5 | 2.5 | 2.5 | 2.5 | 2.5 | 2.5 | 2.5 | 2.5 | 2.5 |
| 1M mono + 1M dibasic at pH 7.5 ml | 52 | 52 | 52 | 52 | 52 | 52 | 52 | 52 | 52 | 52 |
| maltodextrin g | 25 | 36 | 25 | 25 | 36 | 36 | 36 | 36 | 25 | 25 |
| b-cyclodextrin g | 2 | 2 | 2 | 2 | 2 | 2 | 2 | 2 | 2 | 2 |
| sucrose 50% wt/vol ml | 35 | 35 | 35 | 35 | 35 | 35 | 35 | 35 | 35 | 35 |
| tween 80 100% soln ml | 0.6 | 0.6 | 0.6 | 0.6 | 0.6 | 0.6 | 0.6 | 0.6 | 0.6 | 0.6 |
| placebo formulation buffer ml | 40 | 40 | 40 | 40 | 40 | 40 | 40 | 40 | 40 | 40 |
| solid excipients g | 54.5 | 65.5 | 54.5 | 54.5 | 65.5 | 65.5 | 65.5 | 65.5 | 54.5 | 54.5 |
| total feed g | 268 | 279 | 830 | 417 | 279 | 279 | 279 | 279 | 268 | 268 |
| wt % solids | 20% | 23% | 7% | 13% | 23% | 23% | 23% | 23% | 20% | 20% |

TABLE 3
Placebo Feed Formulations 11-20

| Formulation No. | 11 | 12 | 13 | 14 | 15 | 16 | 17 | 18 | 19 | 20 |
|---|---|---|---|---|---|---|---|---|---|---|
| 2% milk 9% solid ml | | | | | | | | | | |
| wfi water ml | 110 | 110 | 110 | 110 | 110 | 110 | 110 | 110 | 110 | 110 |
| 1M mgcl2 ml | 2.5 | 2.5 | 2.5 | 2.5 | 2.5 | 2.5 | 2.5 | 2.5 | 2.5 | 2.5 |
| 1M mono + 1M dibasic at pH 7.5 ml | 52 | 52 | 52 | 52 | 52 | 52 | 52 | 52 | 52 | 52 |
| mannitol g | | 25 | 25 | 39.29 | 25 | 25 | | | | |
| lactose g | | | | | | | 25 | 25 | 25 | 31 |
| maltodextrin g | 25 | | | | | | | | | |
| b-cyclodextrin g | 2 | 2 | 2 | 2 | 2 | 2 | 2 | 2 | 2 | 2 |
| sucrose 50% wt/vol ml | 35 | 35 | 35 | 35 | 35 | 35 | 35 | 35 | 35 | 35 |
| tween 80 100% soln ml | 0.6 | 0.6 | 0.6 | 0.6 | 0.6 | 0.6 | 0.6 | 0.6 | 0.6 | 0.6 |
| triethyl citrate ml | | | | 8.93 | 8.93 | 8.93 | | | | |
| talc g | | | | 1.79 | 5.37 | 5.37 | | | | |
| placebo formulation buffer ml | 40 | 40 | 40 | 40 | 40 | 40 | 40 | 40 | 40 | 40 |
| solid excipients g | 54.5 | 54.5 | 54.5 | 80.7 | 54.5 | 54.5 | 54.5 | 54.5 | 54.5 | 60.5 |
| total feed g | 268 | 268 | 268 | 293 | 282 | 282 | 268 | 268 | 268 | 274 |
| wt % solids | 20% | 20% | 20% | 28% | 19% | 19% | 20% | 20% | 20% | 22% |

TABLE 4
Placebo Feed Formulations 21-30

| Formulation No. | 21 | 22 | 23 | 24 | 25 | 26 | 27 | 28 | 29 |
|---|---|---|---|---|---|---|---|---|---|
| 2% milk 9% solid ml | | | | | | | | | |
| wfi water ml | 110 | 149 | 149 | 149 | 149 | 149 | 149 | 149 | 110 |
| 1M mgcl2 ml | 2.5 | 1.25 | 1.25 | 1.25 | 1.25 | 1.25 | 1.25 | 1.25 | 1.25 |
| 1M mono + 1M dibasic at pH 7.5 ml | 52 | 42 | 42 | 42 | 42 | 42 | 42 | 42 | 52 |
| eudragit susp 2.5, l30d55 30% wt/vol ml | | 28 | 28 | 28 | 47 | 28 | 56.5 | 50 | |
| Raffinose g | | | | | | | | | 2.5 |
| lactose g | 35.7 | | | | | | | | |
| maltodextrin g | | 15 | 15 | 15 | 15 | 15 | 15 | | |
| b-cyclodextrin g | 2 | 2 | 2 | 2 | 2 | 2 | 2 | | |
| sucrose 50% wt/vol ml | 35 | 7.5 | 7.5 | 7.5 | 7.5 | 7.5 | 7.5 | | |
| tween 80 100% soln ml | 0.6 | 0.6 | 0.6 | 0.6 | 0.6 | 0.6 | 0.6 | | |
| triethyl citrate ml | 8.93 | | | | | | | | |
| talc g | 5.37 | | | | | | | | |
| gelatin g | | | | | | | | | 3 |
| placebo formulation buffer ml | 40 | 40 | 40 | 40 | 40 | 40 | 40 | | 40 |
| solid excipients g | 80 | 37.5 | 37.5 | 37.5 | 43.2 | 37.5 | 46.05 | 23 | 15 |
| total feed g | 292 | 285 | 285 | 285 | 304 | 285 | 313.5 | 242 | 210 |
| wt % solids | 27% | 13% | 13% | 13% | 14% | 13% | 15% | 10% | 7% |

TABLE 5
Placebo Feed Formulations 31-34

Formulation No.

| | 30 | 31 |
|---|---|---|
| 2% milk 9% solid ml | 110 | 110 |
| wfi water ml | | |
| 1M mgcl2 ml | | |
| 1M mono + 1M dibasic at pH 7.5 ml | | |
| eudragit susp 2.5, I30d55 30% wt/vol ml | | |
| Raffinose g | | |
| maltodextrin g | 7.5 | 39 |
| b-cyclodextrin g | | |
| sucrose 50% wt/vol ml | | |
| tween 80 100% soln ml | | |
| placebo formulation buffer ml | 40 | 40 |
| solid excipients g | 18.2 | 49 |
| total feed g | 157.5 | 179 |
| wt % solids | 12% | 27% |

Conditions employed in spray drying compositions 1-34 are in Tables 6-7 below:

TABLE 6
Process Conditions for Placebo Formulation Drying

| Formu- | Concen- | Inlet | Outlet | Atom. | Atom. | | Total |
|---|---|---|---|---|---|---|---|
| lation | tration | T | T | Pres | Ra | Airflow | Spray |
| (ID) | (%) | (C.) | (C.) | (bar) | (kg/hr) | (kg/hr) | (g) |
| 1 | 20 | 48 | 25 | 1.2 | 3.0 | 30 | 285.72 |
| 2 | 23 | 48 | 26 | 1.8 | 4.0 | 30 | 267.10 |
| 3 | 7 | 46 | 24 | 1.8 | 4.0 | 30 | 203.30 |
| 4 | 17 | 40 | 21 | 1.2 | 3.0 | 30 | 247.89 |
| 5 | 23 | 40 | 21 | 1.8 | 4.0 | 30 | 245.10 |
| 6 | 23 | 40 | 21 | 1.5 | 3.5 | 30 | 206.44 |
| 7 | 23 | 40 | 22 | 1.3 | 3.0 | 30 | 258.04 |
| 8 | 23 | 40 | 24 | 1.0 | 2.7 | 30 | 260.57 |
| 9 | 20 | 35 | 21 | 1.2 | 3.0 | 30 | 231.92 |
| 10 | 20 | 35 | 21 | 0.9 | 2.5 | 30 | 182.56 |
| 11 | 20 | 35 | 23 | 1.2 | 3.0 | 30 | 77.03 |
| 12 | 20 | 35 | 21 | 1.2 | 3.0 | 30 | 160.03 |
| 13 | 20 | 40 | 34 | 1.2 | 3.0 | 30 | 162.12 |
| 14 | 28 | 35 | 21 | 1.2 | 3.0 | 30 | 118.11 |
| 15 | 19 | 35 | 21 | 1.2 | 3.0 | 30 | 125.60 |
| 16 | 19 | 40 | 24 | 1.1 | 3.0 | 30 | 68.45 |
| 17 | 20 | 35 | 20 | 1.2 | 3.0 | 30 | 113.42 |
| 18 | 20 | 32 | 22 | 1.1 | 3.0 | 30 | 96.01 |
| 19 | 20 | 35 | 25 | 0.9 | 2.5 | 30 | 146.82 |
| 20 | 22 | 35 | 25 | 0.9 | 2.5 | 30 | 146.11 |
| 21 | 27 | 35 | 25 | 0.9 | 2.5 | 30 | 81.65 |
| 22 | 13 | 35 | 25 | 1.2 | 3.0 | 30 | 99.05 |
| 23 | 13 | 35 | 25 | 0.9 | 2.5 | 30 | 73.41 |
| 24 | 13 | 30 | 22 | 1.2 | 3.0 | 30 | 67.15 |
| 25 | 14 | 30 | 21 | 1.2 | 3.0 | 30 | 105.19 |
| 26 | 13 | 30 | 21 | 1.3 | 3.0 | 30 | 89.19 |
| 27 | 15 | 30 | 21 | 1.3 | 3.0 | 30 | 93.95 |
| 28 | 10 | 30 | 21 | 1.3 | 3.0 | 30 | 144.66 |
| 29 | 7 | 30 | 21 | 1.3 | 3.0 | 30 | 223.41 |
| 30 | 17 | 30 | 21 | 1.3 | 3.0 | 30 | 76.31 |
| 31 | 14 | 30 | 20 | 1.2 | 3.0 | 30 | 109.37 |
| 32 | 12 | 30 | 21 | 1.1 | 3.0 | 30 | 63.81 |
| 33 | 27 | 64 | 35 | 1.1 | 3.0 | 30 | 88.44 |
| 34 | 23 | 30 | 22 | 1.1 | 3.0 | 30 | 68.23 |

TABLE 7
Process Conditions for Placebo Formulation Drying

| | Spray | Run | Cyclone no | Cyclone | Total | Particle | % |
|---|---|---|---|---|---|---|---|
| Formulation | Rate | Time | hammering | hammering | Yield | Size | Moisture |
| (ID) | (g/min) | (min) | (g) | (g) | (%) | (micron) | (%) |
| 1 | 5.05 | 56.6 | 13.03 | | 22.80 | | |
| 2 | 5.33 | 50.1 | 3.74 | 23.30 | 44.02 | | |
| 3 | 4.78 | 42.5 | 0.32 | | 2.25 | | |
| 4 | 4.28 | 57.9 | 0.00 | | 0.00 | | |
| 5 | 4.30 | 57.0 | 8.88 | 13.97 | 40.53 | 9.278 | 7.65 |
| 6 | 4.38 | 47.1 | 3.69 | 14.96 | 39.28 | 9.446 | 8.02 |
| 7 | 4.29 | 60.2 | 6.74 | 18.44 | 42.43 | 7.436 | 8.20 |
| 8 | 4.32 | 60.3 | 8.12 | 13.00 | 35.24 | 7.798 | 7.55 |
| 9 | 3.80 | 61.0 | 0.55 | 18.07 | 40.14 | 9.364 | 6.81 |
| 10 | 3.71 | 49.2 | 0.26 | 7.82 | 22.13 | | 7.67 |
| 11 | 3.02 | 26.6 | 0.10 | | 0.65 | | |
| 12 | 3.77 | 42.4 | 0.72 | | 2.25 | | |
| 13 | 4.32 | 37.5 | 0.32 | | 0.99 | | |
| 14 | 3.91 | 30.2 | 0.42 | | 1.27 | | |
| 15 | 3.99 | 31.5 | 0.19 | | 0.80 | | |
| 16 | 4.39 | 15.6 | 0.20 | | 1.54 | | |
| 17 | 3.69 | 30.7 | 0.50 | | 2.20 | | |
| 18 | 2.44 | 39.4 | 0.97 | | 5.05 | | |
| 19 | 2.44 | 60.1 | 2.04 | 10 | 41.00 | 13.225 | 4.64 |
| 20 | 2.76 | 63.0 | 6.60 | 4.46 | 40.69 | 10.817 | 4.85 |
| 21 | 2.66 | 30.7 | 0.50 | 9.33 | 44.59 | | 4.92 |
| 22 | 2.20 | 45.0 | 4.89 | 1.32 | 48.23 | 9.808 | 4.83 |
| 23 | 2.22 | 33.1 | 3.44 | 0.84 | 44.85 | 6.928 | 6.63 |
| 24 | 2.15 | 40.6 | 4.49 | 1.33 | 51.37 | 7.168 | 4.42 |
| 25 | 2.26 | 46.6 | 6.42 | 0.38 | 46.17 | 7.07 | 4.62 |
| 26 | 2.22 | 40.2 | 4.90 | 0.23 | 44.24 | 6.838 | 4.75 |
| 27 | 2.33 | 40.4 | 5.74 | 0.44 | 43.85 | 7.022 | 4.81 |
| 28 | 2.33 | 62.1 | 4.07 | 0.32 | 30.35 | 8.209 | 4.62 |
| 29 | 2.31 | 96.6 | 7.40 | 1.35 | 65.95 | 6.398 | 4.42 |
| 30 | 2.27 | 33.6 | | | 0.00 | | |
| 31 | 2.18 | 50.1 | | | 0.00 | | |
| 32 | 1.94 | 32.9 | | | 0.00 | | |
| 33 | 2.22 | 39.9 | | | 0.00 | | |
| 34 | 2.29 | 29.8 | | | 0.00 | | |

As shown in tables 2-7 several successful formulations were spray dried at different compositions, with solids varying from 7% to 27% in feed and various process parameters with inlet temperatures as low as 30° C. (range of 48 to 30° C.) and outlet temperature as low as 20° C. (20° C. to 35° C.)

### Enteric Feed Preparation

Eudragit or enteric coating formulations were produced where feed preparation is an important component to the spray drying process. Eudragit L30D55 solution is added to buffer containing a mixture of mono and dibasic phosphate with a pH of 7.4 to 7.6 and a low concentration of magnesium chloride. As the solution turns opalescent, indicating that the Eudragit is solubilizing, the pH drops below 7. Preparation of the feed in this fashion is important in certain cases, as when virus stock is added to the buffer containing soluble enteric coating, virus will be coated during the spray drying process. The enteric coating solution needs to be homogeneous (e.g., the enteric polymer should be completely dissolved), and that could be achieved if phosphate buffer of pH 7.4 or higher is used. If the phosphate buffer of lower pH is used, there may be insoluble Eudragit polymer which may flocculate or aggregate in the feed formulation, and as a result, may not coat the virus during a spray drying process. In addition, insoluble or flocculated components may clog the atomization nozzle in the spray-drying apparatus.

### Nozzle Shear

Addition of surfactant during the feed preparation is also important in certain cases, since surfactant can reduce the shear force between the atomizing nozzle and the virus in the feed during the spray drying process. Typically during the feed preparations surfactant is added once all salts and sugars are completely solubilized. Adding surfactant before beta cyclodextrin is completely solubilized can form insoluble complexes between beta cyclodextrin and the surfactant(s). Once such complexes are formed the stability effect of beta cyclodextrin in the virus formulation may be diminished and the shear force reduction provided by the surfactant may be lost. Thus, avoiding the formation of insoluble complexes in the feed preparation may be important for viable virus in the resultant spray dried powders.

Alternatively, feed preparations may be prepared such that the viral particles are effectively “trapped” in complexes or flocculated particles formed. In such formulations, the exposure of virus to shear forces associated with spray drying may be attenuated due to the encapsulation effect.

### Virus Formulation Testing

Adenovirus 4 Live Virus formulations with various pharmaceutical excipients (including maltodextrin, lactose, sucrose, β-cyclodextrin and Eudragit L30-D55) in DI Water were tested. Test Objectives were to use process conditions developed during previous test work with placebo (supra) to produce powder samples containing live virus for analysis to determine virus survival rate during the spray drying process.

Sixteen runs were performed in nine days of testing on A SD-Micro spray dryer located inside a biosafety cabinet in the GEA Niro A/S Test Facility in Copenhagen. Ten powder samples were produced using formulations containing live virus.

### Equipment and Process Set-Up

GEA Niro SD-Micro Spray Dryer
Insulated Stainless Steel Drying Chamber, cyclone, and ductwork (placebo)
Standard glass chamber (virus)
Niro two-fluid nozzle w/0.5 mm liquid insert
2.4 mm ID, 3.2 mm OD Silicone tubing
Watson Marlow 505 L Peristaltic Pump
Filter cleaning=manual

The SD-Micro was selected based on the quantities of material available, and the ability to use Nitrogen as the process gas. Reasons to use Nitrogen as the drying gas include processing organic solvents or for product stability concerns. Using Nitrogen in a single-pass configuration (see FIG. 3) has the additional benefits of yielding low residual water levels in the powders that are produced, as the inlet compressed Nitrogen has a very low dew point. The Two-Fluid Nozzle was selected due to the laboratory scale of spray drying, and the ease of use and adjustability.

Liquid feeds were prepared and transferred inside the biosafety cabinet through an airlock. Once inside, the live virus was added (after thawing), and an initial liquid sample was taken and removed from the biosafety cabinet and placed in the −80 degrees C. freezer. Near the completion of the run, an endpoint liquid sample was also taken; these and all powder samples were placed in the freezer as soon as possible.

Powder samples were collected below the cyclone in 100 ml, Kimax media bottles. Samples were generally collected at the end of runs, or at 1 hour time points during the runs. At the completion of the run, the chamber, ductwork, and cyclone were mechanically agitated to free material which was loosely adhered, and this material was segregated as a separate sample. All sample vials and jars which were removed from the biosafety cabinet were first wiped down with bleach rags. The entire inside of the isolator and spray dryer were cleaned and decontaminated at the conclusion of the trials.

The testing began using the feed formulations and process parameters which had been previously developed using the stainless steel chamber and insulated ductwork to ensure that the two spray dryers functioned the same before adding the virus. Although the process parameters were the same, due to the restricted access of the biosafety cabinet, it was decided that for the critical virus runs, it was desirable to be able to visually see the spray pattern and chamber deposit level. Additionally, the process outlet temperature was approximately the same temperature as the room temperature, so the net benefit of the insulated stainless steel chambers and ductwork was thought to be minimal, and therefore the glass chambers were used for all live virus trials.

The live virus testing went very well, and Runs #7, 8, 9, and 10 were the samples comprising either maltodextrin or lactose, and then Eudragit with and without maltodextrin. The two runs without Eudragit had total yields in the range of 69-74%, which is very good for relatively short runs at very low temperatures. The Eudragit runs had total yields in the 29-34% range. In this case total yield is defined as the yield with and without mechanical agitation, and from the Process Parameter summary in Table 10, below, the highest yield without agitation was maltodextrin (Run #7), followed by lactose (Run #8), and finally the lowest was the Eudragit (Runs #9 and 10). Additionally, from the Eudragit runs, Run #9 with maltodextrin had a higher yield than Run #10 with no maltodextrin. The reason for this may be that maltodextrin is slightly better suited to spray drying than lactose (because maltodextrin has a higher Tg), and therefore the yield may be slightly better. The Eudragit appears to not be nearly as good as either maltodextrin of lactose, because of the lower Tg and higher feed solution viscosity, and therefore the yield goes down with the higher Eudragit level. Finally, both maltodextrin and lactose adhere very loosely when they form deposits in the chamber and ductwork, and when they are mechanically agitated tend to come loose easily, while Eudragit as a polymer adheres when it forms deposits, and therefore almost none comes free when agitated. It is possible that the yield from Eudragit runs would improve upon scale-up to a larger spray dryer, as there are more types of atomizers that are available at larger scale.

The formulations used for the testing are reported in Table 9, below. All feed preparation work was performed by using magnetic stir plates and typical laboratory supplies. Process parameters are reported in Table 10, below. All samples were collected in 100 mL Kimax media bottles.

TABLE 8
Virus Feed Formulations 1-10

| Formulation | 1 | 2 | 3 | 4 | 5 | 6 | 7 | 8 | 9 | 10 |
|---|---|---|---|---|---|---|---|---|---|---|
| 2% milk 9% solid | | | | | | | | | | |
| wfi water ml | 110 | 110 | 110 | 110 | 149 | 149 | 220 | 220 | 298 | 298 |
| 1M mgcl2 ml | 2.5 | 2.5 | 2.5 | 2.5 | 1.25 | 1.25 | 5 | 5 | 2.5 | 2.5 |
| 1M mono + 1M dibasic at pH 7.5 ml | 52 | 52 | 52 | 52 | 52 | 42 | 104 | 104 | 104 | 84 |
| eudragit suspH 2.5, l30d55 30% wt/vol ml | | | | | 47 | 50 | | | | |
| lactose g | | 31 | | 31 | | | | 62 | | |
| maltodextrin g | 36 | | 36 | | 15 | | 72 | | 30 | |
| b-cyclodextrin g | 2 | 2 | 2 | 2 | 2 | | 4 | 4 | 4 | |
| sucrose 50% wt/vol ml | 35 | 35 | 35 | 35 | 7.5 | | 70 | 70 | 15 | |
| tween 80 100% soln ml | 0.6 | 0.6 | 0.6 | 0.6 | 0.6 | | 1.2 | 1.2 | 1.2 | |
| virus formulation PXVX1013 VECTOR | 40 | 20 | 40 | 20 | 10 | 10 | 40 | 40 | 40 | 40 |
| solid excipients g | 65.5 | 60.5 | 65.5 | 60.5 | 43.2 | 23 | 121 | 121 | 88 | 46 |
| total feed g | 279 | 254 | 279 | 254 | 284 | 252 | 518 | 548 | 588 | 525 |

TABLE 9
Process Conditions for Virus Formulation Drying

| | Formu- | Concen- | Inlet | Outlet | Atom. | Atom. | Nitro- |
|---|---|---|---|---|---|---|---|
| | lation | tration | Temp. | Temp. | Press. | Rate | gen |
| Run | (D) | (%) | (C.) | (C.) | (bar) | (kg/hr) | (kg/hr) |
| 1 | 1 | 23 | 40 | 21 | 1.3 | 3.0 | 30 |
| 2 | 2 | 24 | 40 | 21 | 1.3 | 3.0 | 30 |
| 3 | 1 | 23 | 40 | 26 | 0.9 | 2.5 | 30 |
| 4 | 2 | 24 | 40 | 24 | 0.9 | 2.5 | 30 |
| 5 | 3 | 15 | 42 | 23 | 1.3 | 3.0 | 30 |
| 6 | 4 | 9 | 30 | 21 | 1.3 | 3.0 | 30 |
| 7 | 1 + | 23 | 35 | 23 | 2.1 | 4.0 | 30 |
| | Virus | | | | | | |
| 8 | 2 + | 22 | 35 | 22 | 2.2 | 4.0 | 30 |
| | Virus | | | | | | |
| 9 | 3 + | 15 | 34 | 20 | 2.2 | 4.0 | 30 |
| | Virus | | | | | | |
| 10 | 4 + | 9 | 34 | 20 | 2.2 | 4.0 | 30 |
| | Virus | | | | | | |

TABLE 10
Process Conditions for Virus Formulation Drying

| | | | | Cyclone | Cylone | | |
|---|---|---|---|---|---|---|---|
| | Total | Spray | Run | no ham- | ham- | Total | |
| | Sprayed | Rate | Time | mering | mering | Yield | |
| Run | (g) | (g/min) | (min) | (g) | (g) | (%) | Comments |
| 1 | 265 | 6.80 | 39 | — | — | N/A | Placebo |
| 2 | 241 | 7.31 | 33.0 | — | — | N/A | Placebo |
| 3 | 265 | 13.95 | 19.0 | — | — | N/A | Placebo |
| 4 | 241 | 9.28 | 26.0 | — | — | N/A | Placebo |
| 5 | 270 | 8.43 | 32.0 | — | — | N/A | Placebo |
| 6 | 239 | 13.30 | 18.0 | — | — | N/A | Placebo |
| 7 | 492 | 3.35 | 147.0 | 63.50 | 20.50 | 74.22 | |
| 8 | 521 | 3.83 | 136.0 | 31.50 | 48.00 | 69.41 | |
| 9 | 559 | 3.10 | 180.0 | 28.00 | 0.50 | 34.01 | |
| 10 | 499 | 3.42 | 146.0 | 13.00 | 0.00 | 28.96 | |

### Large Scale Testing

Spray drying tests at larger (pilot) scale using formulations as previously described herein were performed. Larger samples were produced to demonstrate potential throughput. Low-temperature process conditions previously shown to have a favorable effect on the virus survival rate were employed.

Tests were performed on the MOBILE MINOR spray dryer. In one of the tests, 870 grams of dry powder was produced in a single five-hour period. Some of the formulations did not perform well, and it was later confirmed on the SD-Micro that these are not good formulations in general for low-temperature spray drying. In general:
Cyclone yields were comparable to the SD-Micro (45-55%). Total overall yields of 89% were obtained. Cyclone yields can be optimized for higher total yield.
Low outlet temperatures between 32-35 degrees C. were demonstrated to be feasible with relatively low spray rates (˜12-13 g/min).
10% Solutions containing Eudragit were processed using a high atomization setpoint (12.6 kg/hr) and solutions without Eudragit were processed using a relatively low setpoint (7.8 kg/hr).

Equipment Set-Up:
Niro MOBILE MINOR 2000 Spray Dryer
Insulated Stainless Steel Drying Chamber and Baghouse
Niro two-fluid nozzle w/1.0 mm liquid insert
2.4 mm ID, 3.2 mm OD Marprene tubing (no “Y”)
Watson Marlow 505 L Peristaltic Pump
Filter cleaning=manual
Nitrogen purge on the cyclone collection jar was installed, regulated at 2.0 psig and 30 LPM, and was approximately 18 degrees C. with a dewpoint of −12 degrees C.

The embodiments described herein and illustrated by the foregoing examples should be understood to be illustrative of the present invention, and should not be construed as limiting. On the contrary, the present disclosure embraces alternatives and equivalents thereof, as embodied by the appended claims.

What is claimed:

1. A formulation for spray-drying comprising viral particles, sucrose, and cyclodextrin.

2. The formulation of claim 1, wherein said viral particles are adenoviral particles.

3. The formulation of claim 1, wherein said viral particles comprises an adenoviral vector that is replication competent.

4. The formulation of claim 1, wherein the cyclodextrin is α-cyclodextrin, β-cyclodextrin, γ-cyclodextrin or a mixture thereof.

5. The formulation of claim 1, wherein the total amount of sucrose and cyclodextrin is from about 5 wt % to about 15 wt % of said formulation.

6. The formulation of claim 1 further comprising an excipient.

7. The formulation of claim 1 further comprising raffinose, mannitol, lactose, starch or a mixture thereof.

8. The formulation of claim 1 further comprising a spray aid.

9. The formulation of claim 1 further comprising maltodextrin, lactose, talc, triethyl citrate, gelatin or a mixture thereof.

10. The formulation of claim 1 further comprising a spray aid in an amount from about 3 wt % to about 15 wt %.

11. The formulation of claim 1 further comprising a surfactant.

12. The formulation of claim 1 further comprising polysorbates, poloxamers or a mixture thereof.

13. The formulation of claim 1 further comprising a surfactant in an amount from about 0.05 wt % to about 1 wt %.

14. The formulation of claim 1 is an aqueous formulation.

15. The formulation of claim 1, wherein the total solids content of the formulation is less than about 30 wt %.

16. The formulation of claim 1, wherein the total solids content of the formulation is from about 5 wt % to about 25 wt %.

17. A spray-dried powder comprising viral particles, sucrose and cyclodextrin.

18. A formulation for spray drying comprising viral particles and a carbohydrate, wherein the total solids content of the formulation is less than about 30 wt %.

19. The formulation of claim 18, wherein the carbohydrate is sucrose, α-cyclodextrin, β-cyclodextrin, γ-cyclodextrin, raffinose, mannitol, lactose, starch or a mixture thereof.

20. The formulation of claim 18 further comprising a surfactant.

21. A formulation for spray drying comprising viral particles, a carbohydrate and an enteric polymer.

22. The formulation of claim 21, further comprising a surfactant.

23. The formulation of claim 21, wherein the enteric polymer is Eudragit® L 30 D-55.

24. A spray-dried powder comprising viral particles, a carbohydrate and an enteric polymer.

25. A method for large scale spray drying comprising spraying drying the formulation of claim 1, 18 or 21 at an outlet temperature of less than about 40° C.

26. The method of claim 25, wherein the outlet temperature is less than about 30° C.

27. A spray-dried powder produced by the method of claim 25.

28. A spray-dried powder made from the formulation of claim 1, 18 or 21.

29. The spray-dried powder of claim 27, wherein the viral activity of the viral particles is at least about 70% of the viral activity prior to spray drying.

30. A pharmaceutical composition for oral administration comprising the spray-dried power of claim 27.

31. A vaccine comprising the spray-dried powder of claim 27.

32. A replication competent vaccine for oral administration comprising the spray-dried powder of claim 27.

33. The spray-dried powder of claim 28, wherein the viral activity of the viral particles is at least about 70% of the viral activity prior to spray drying.

34. A pharmaceutical composition for oral administration comprising the spray-dried power of claim 28.

35. A vaccine comprising the spray-dried powder of claim 28.

36. A replication competent vaccine for oral administration comprising the spray-dried powder of claim 28.
